(19) United States
(12) Patent Application Publication
(10) Pub. No.: US 2023/0245730 A1
(43) Pub. Date: Aug. 3, 2023

(54) METHOD FOR EVALUATING BIODEGRADABILITY OF SEWAGE THROUGH MACHINE LEARNING

(71) Applicant: Nanjing University, Nanjing (CN)

(72) Inventors: Haidong HU, Nanjing (CN); Huazai CHENG, Nanjing (CN); Bing WU, Nanjing (CN); Hongqiang REN, Nanjing (CN)

(21) Appl. No.: 17/588,223

(22) Filed: Jan. 29, 2022

(51) Int. Cl. G16C 60/00; G01N 33/18; G16C 20/30; G16C 20/70

(52) U.S. Cl. CPC G16C 60/00; G01N 33/1826; G16C 20/30; G16C 20/70

(57) ABSTRACT

A method for evaluating the biodegradability of sewage through machine learning, includes: (1) collecting molecular composition information and biodegradability data of organic molecules in a sewage sample; (2) establishing a model for predicting biodegradability of organic molecules in sewage through machine learning; (3) measuring the molecular composition information of organic molecules in sewage from a target sewage plant; and (4) predicting, according to the model established in (2), the biodegradability of the organic molecules in the sewage from the target sewage plant.

### BACKGROUND

The disclosure relates to the field of sewage treatment, and more particularly to a method for evaluating the biodegradability of sewage through machine learning.

Sewage is a type of wastewater that contains a large amount of dissolved organic matter, some of which can be degraded and utilized by microorganisms, while others are generally difficult to biodegrade and even inhibit the growth of microorganisms. Biodegradability of sewage, which is one of the characteristics of sewage, refers to the proportion of biodegradable organic matter in the total organic matter. Thus, evaluating the biodegradability may be a consideration for sewage treatment processes.

Currently, BOD₅/COD relationship allows the biodegradability of the sewage to be assessed. COD (Chemical oxygen demand) of sewage is generally measured by the potassium dichromate method. A basic analysis method for measuring BOD₅ includes: adding sufficient sewage (or dilution sewage) to a glass bottle so that a stopper can be inserted without leaving air; placing the glass bottle in an incubator at 20° C. ± 1° C. for five days; measuring the dissolved oxygen concentration in the sewage after the incubation period; and calculating the difference between the initial and final dissolved oxygen concentrations to obtain the BOD of the sewage. When certain microorganisms are absent in some industrial wastewater, they should be inoculated into the industrial wastewater before BOD measurement. Thus, the conventional method for evaluating the biodegradability of the sewage is a time-consuming and tedious process.

### SUMMARY

To solve the aforesaid problems, the disclosure provides a method for evaluating the biodegradability of sewage through machine learning, the method comprising:
(1) collecting molecular composition information and biodegradability data of organic molecules in a sewage sample;
(2) establishing a model for predicting biodegradability of organic molecules in sewage through machine learning;
(3) measuring the molecular composition information of organic molecules in sewage from a target sewage plant; and
(4) predicting, according to the model established in (2), the biodegradability of the organic molecules in the sewage from the target sewage plant.

In a class of this embodiment, the molecular composition information of organic molecules in the sewage sample comes from data measured by a Fourier transform ion cyclotron resonance mass spectrometer, and the biodegradability of the organic molecules in the sewage is represented by BOD₅/COD.

In a class of this embodiment, the model for predicting the biodegradability of organic molecules in the sewage is established by a multi-layer perceptron that is a neural network model used in machine learning, which comprises:
(a) calculating a molecular parameter of the organic molecules, and performing data standardization by using the molecular parameter as a feature value;
(b) calculating the Pearson correlation coefficient between the feature values and the biodegradability of the organic molecules; extracting, according to the absolute value of the Pearson correlation coefficient, desired feature values as input features in a neural network;
(c) splitting a dataset into a training set and a test set, determining topology of the neural network, namely determining the number of hidden layer and the number of neurons in each hidden layer; and
(d) optimizing hyperparameters of the model, training the neural network with the training set, and evaluating the performance of the neural network by using the test set.

In a class of this embodiment, in (a), the molecular parameter as the feature value comprises: the molecular parameters of all organic molecules, and the molecular parameters of seven classes of organic molecules.

In a class of this embodiment, the molecular parameters of all organic molecules comprise: a mass-to-charge ratio m/z, a number C of carbon atoms, a number H of hydrogen atoms, a number O of oxygen atoms, a number N of nitrogen atoms, a ratio O/C of the number of oxygen atoms to the number of carbon atoms, a ratio H/C of the number of hydrogen atoms to the number of carbon atoms, a number DBE of double bond equivalents, a ratio DBE/H of the number of double bond equivalents to the number of hydrogen atoms, a ratio DBE/O of the number of double bond equivalents to the number of oxygen atoms, a ratio (DBE-O)/C of a difference between the number of double bond equivalents and the number of oxygen atoms to the number of carbon atoms, an average value of a nominal oxidation state of carbon (NOSC) of all organic molecules, and strength weighted average values of molecular parameters, which are equal to a sum of products of respectively multiplying corresponding relative peak strength of molecules by m/z, C, H, O, N, O/C, H/C, DBE, DBE/H, DBE/O, (DBE-O)/C and NOSC.

In a class of this embodiment, the seven classes of organic molecules are: lipids, proteins/amino sugars, carbohydrates, unsaturated hydrocarbons, lignin, tannins and condensed aromatics; the screening conditions for lipids are as follows: O/C < 0.2 and 1.7 < H/C < 2.2; the screening conditions for proteins/amino sugars are as follows: 0.2 < O/C < 0.6, 1.5 < H/C < 2.2 and N/C ≥ 0.05; the screening conditions for carbohydrates are as follows: 0.6 < O/C < 1.0 and 1.5 < H/C < 2.2; the screening conditions for unsaturated hydrocarbons are as follows: O/C<0.1, 0.7<H/C<1.5; the screening conditions for lignin are as follows: 0.1 < O/C < 0.6, 0.6 < H/C < 1.7, and the modified aromaticity index AImod < 0.67; the screening conditions for tannins are as follows: 0.6 < O/C < 1.0, 0.5 < H/C < 1.5 and the modified aromaticity index AImod < 0.67; and, the screening conditions for condensed aromatics are as follows: O/C < 1.0, 0.3 < H/C < 0.7 and the modified aromaticity index AImod ≥ 0.67.

In a class of this embodiment, the molecular parameters of seven classes of organic molecules comprise: the mass-to-charge ratio m/zi, the number DBEi of double bond equivalents, and the average value of the nominal oxidation state of carbon NOSCi of seven classes of organic molecules, the proportion Numi of the number of molecules in each class, and strength weighted average values of molecular parameters, which are equal to a sum of products of respectively multiplying corresponding relative peak strength of molecules by m/zi, DBEi and NOSCi, which i represents the molecule classes.

In a class of this embodiment, in (a), the data standardization is performed on the feature values using the formula:

$$z = \frac{\left(x - u\right)}{s}$$

where z is the standardized feature value, x is the original feature value, u is the average value of the features, and s is the standard deviation of the feature values.

In a class of this embodiment, in (b), the Pearson correlation coefficient between the feature value and the biodegradability of the organic molecules is calculated using the formula:

$$r = \frac{{\sum }_{i = 1}^{n} \left({x}_{i} - \overline{x}\right) \left({y}_{i} - \overline{y}\right)}{\sqrt{{\sum }_{i = 1}^{n} {\left({x}_{i} - \overline{x}\right)}^{2}} {\sum }_{i = 1}^{n} {\left({y}_{i} - \overline{y}\right)}^{2}}$$

where xᵢ is the feature value, yᵢ is the measured value of the biodegradability of the organic molecules in the sewage,

$$\overline{x} = \frac{1}{n} {\sum }_{i = 1}^{n} {x}_{i} , \overline{y} =$$

$$\frac{1}{n} {\sum }_{i = 1}^{n} {y}_{i} ,$$

n is the total number of sewage samples. A correlation matrix of the feature value and the biodegradability of the organic molecules in the sewage are obtained using the formula. According to the absolute value of the correlation coefficients, the feature values highly correlated with the biodegradability of the organic molecules in the sewage are selected as input values of the neural network.

In a class of this embodiment, in (c), the dataset is randomly split into a training set and a test set in a ratio of 7: 3. A model for biodegradability forecasting is established based on the multi-layer perceptron. The input value of the input layer is connected to the neurons in the hidden layer and the neurons in the hidden layer are connected to the neurons of the output layer. Each neuron in one layer is connected to all neurons in the next layer. The topology of the multi-layer perceptron is determined as follows: the range for the numbers of the neurons in each hidden layer is determined according to the number of input variables; the range for the number of the hidden layers is determined according to the characteristics of data structure, and the same number of neurons is used for all hidden layers.

In a class of this embodiment, in (c), the topology of the multi-layer perceptron comprises m input neurons, n hidden neurons, and one output neuron. The output of the neural network, namely the predicted value for biodegradability of sewage, can be expressed within the following equation:

$${y}^{⌣} = W θ \left({\sum }_{i = 1}^{m} {w}_{i} {x}_{i} + {b}_{1}\right) + {b}_{2}$$

where, y̅ is the predicted value, W and wᵢ are respectively the weights of hidden layer and input layer; b₁ and b are respectively the bias added for the hidden layer and the output layer; and θ is an activation function.

Training the neural network is to minimize a loss function. The loss function is expressed as below:

$$L o s s = \frac{1}{2} {\left‖{y}^{⌣} - y\right‖}_{2}^{2} + \frac{α}{2} {\left‖W\right‖}_{2}^{2}$$

where,

$$α / 2 {\left‖W\right‖}_{2}^{2}$$

is the L2 regularization term for penalizing a complex model.

The parameters in the opposite direction of the gradient of the objective function are updated at each iteration through gradient descent. For example, an example formula of gradient descent is as follows where the weights are updated:

$$\begin{matrix}{W}^{i + 1} ← {W}^{i} + \text{Δ} {W}^{i} \\ \text{Δ} {W}^{i} = - η ∇ L o s {s}_{W}^{i}\end{matrix}$$

where, i is the number of iterations, η∈(0,1) is the learning rate,

$$∇ L o s {s}_{W}^{i}$$

is the gradient of the loss function with respect to the weights.

The hyderparameters that need to be optimized comprises: an algorithm used to minimize the loss function, comprising stochastic gradient descent (SGD), adaptive moment estimation (Adam) and Limited-memory BFGS (L-BFGS); an activation function, comprising Sigmoid, tanh and ReLU; a parameter α for L2 regularization term; and a maximum number iter of iterations; the training set is used to fit the neural network, and the test set is used to measure the performance of the neural work; the coefficient of determination (R²) and root mean squared error (RMSE) are used as indicators for evaluating the accuracy of the model. R² is calculated using the formula:

$${R}^{2} \left(y , {y}^{⌣}\right) = 1 - \frac{{\sum }_{i = 1}^{n} {\left({y}_{i} - {{y}^{⌣}}_{i}\right)}^{2}}{{\sum }_{i = 1}^{n} {\left({y}_{i} - \overline{y}\right)}^{2}}$$

RMSE is calculated using the formula:

$$R M S E = \sqrt{\frac{1}{n} {\sum }_{i = 1}^{n} {\left({{y}^{⌣}}_{i} - {y}_{i}\right)}^{2}}$$

where, yᵢ is the measured value, y̅ᵢ is the predicted value,

$$\overline{y} = \frac{1}{n} {\sum }_{i = 1}^{n} {y}_{i} , \text{n}$$

is the total number of the sewage samples.

In a class of this embodiment, in (3) and (4), using the model to evaluate the biodegradability of sewage, comprises:
(a) measuring the molecular composition information of organic molecules in the sewage sample by a Fourier transform ion cyclotron resonance mass spectrometer;
(b) extracting a desired feature value; and performing data normalization on the feature value; and
(c) feeding the feature value in (b) into the model, running the model to obtain an output value for the biodegradability of organic molecules in sewage.

The following advantages are associated with the method for evaluating biodegradability of organic molecules in sewage through machine learning of the disclosure.

(1) In the disclosure, few test water samples are required for evaluating the biodegradability of organic molecules in sewage, and a 5-day culture is not necessary, so that the test period can be greatly shortened. In addition, the biodegradability of organic molecules in sewage can be predicted immediately after the molecular composition information of organic molecules is obtained.

(2) In the disclosure, the method for evaluating the biodegradability of organic molecules in sewage is easy to operate, so that the tedious experimental operation processes such as algae biological culture are avoided.

### BRIEF DESCRIPTION OF THE DRAWINGS

FIG. 1 is a histogram showing a frequency distribution of input feature values after applying standardization according to Example 1 of the disclosure;

FIG. 2 is a graph of RMSE versus neural network topology with different layers along with different neurons according to Example 1 of the disclosure;

FIG. 3 is a schematic diagram of a neural network established in Example 1 of the disclosure; and

FIG. 4 is a graph for assessing the performance of a neural network implemented in Example 1 according to the disclosure.

### DETAILED DESCRIPTION

To further illustrate, embodiments detailing a method for evaluating the biodegradability of sewage through machine learning are described below. It should be noted that the following embodiments are intended to describe and not to limit the disclosure.

### Example 1

Sewage samples from a sewage plant are selected to evaluate the biodegradability of organic molecules. The specific evaluation method was described below:

(a) 82 pieces of the molecular composition information of organic molecules in sewage measured by a Fourier transform ion cyclotron resonance mass spectrometer and the biodegradability data are collected.

(b) The molecular parameters of the organic molecules in each sewage sample are calculated as feature values. The specific calculation process is described below.

The molecular parameters of all organic molecules comprise: the average value of the mass-to-charge ratio m/z; the average value of the number C of carbon atoms, the average value of the number H of hydrogen atoms; the average value of the number O of oxygen atoms; the average value of the number N of nitrogen atoms; the average value of the ratio O/C of the number of oxygen atoms to the number of carbon atoms; the average value of the ratio H/C of the number of hydrogen atoms to the number of carbon atoms; the average value of the number of double bond equivalents (DBE) (i.e.,

$$\left(\text{DBE=} \frac{\text{2C+N+P-H+2}}{2}\right) ;$$

the average value of the ratio of the number of double bond equivalents to the number of hydrogen atoms (DBE/H); the average value of the ratio of the number of double bond equivalents to the number of oxygen atoms (DBE/O); the average value of the ratio of the difference between the number of double bond equivalents and the number of oxygen atoms to the number of carbon atoms ((DBE-O)/C) (i.e.,

$$\left(\frac{\text{DBE} - \text{O}}{\text{C}} = \frac{\left(\text{2C+N+P} - \text{H+2}\right) / 2 - \text{O}}{\text{C}}\right) ;$$

the average value of the nominal oxidation state of carbon (NOSC) (i.e.,

$$\left(\text{NOSC} = 4 - \frac{\text{4C+H} - 2 \text{O} - \text{3N} - 2 \text{S+5P}}{\text{C}}\right) ;$$

the sum of strength weighted average values of m/z (i.e., m/z_{wa} = ∑(m/zᵢ × RIᵢ)); the sum of strength weighted average values of C (i.e., C_{wa} = Σ(Cᵢ × RIᵢ)); the sum of strength weighted average values of H (i.e., H_{wa} = Σ(Hᵢ × RIᵢ)); the sum of strength weighted average values of O (i.e., O_{wa} = Σ(Oᵢ × RIᵢ)); the sum of strength weighted average values of N (i.e., N_{wa} = Σ(Nᵢ × RIᵢ)); the sum of strength weighted average values of O/C (i.e., O/C_{wa} = Σ(O/Cᵢ × RIᵢ)); the sum of strength weighted average values of H/C (i.e., H/C_{wa} = Σ(H/Cᵢ × RIᵢ)); the sum of strength weighted average values of DBE (i.e., DBE_{wa} = Σ(DBEᵢ × RIᵢ)); the sum of strength weighted average values of DBE/H (i.e., DBE/H_{wa} = Σ(DBE/Hᵢ × RIᵢ)); the sum of strength weighted average values of DBE/O (i.e., DBE/H_{wa} = Σ(DBE/Hᵢ × RIᵢ)); the sum of strength weighted average values of (DBE-O)/C (i.e., (DBE — O)/C_{wa} = Σ((DBE — O)/Cᵢ × RIᵢ)); and, the sum of strength weighted average values of NOSC (i.e., NOSC_{wa} = Σ(NOSCᵢ × RIᵢ)).

All organic molecules in each sample are classified into seven classes. By taking the calculation process of lipids as an example, the molecular parameters of all molecules in this class are calculated as follows: the average value of m/z; the average value of DBE; the average value of NOSC; the sum of strength weighted average values of m/z; the sum of strength weighted average values of DBE; the sum of strength weighted average values of NOSC; and, the ratio Num₁ of the number of molecules of this class in the number of all molecules in this sample. The calculation process of other six classes is the same as above and would not be repeated here.

(3) 73 features values obtained for each sewage sample are merged, and there are totally 82 sewage samples, so 82 original sample sets (x₁, x₂, x₃, ..., x₈₂) are obtained. Data standardization is performed on the calculated feature values by the following calculation formula:

$$z = \frac{\left(x - u\right)}{s}$$

where z is the standardized feature value, x is the original feature value, u is the average value of the feature value, and s is the standard deviation of the feature value. The data of the biodegradability of organic nitrogen in the sewage samples is incorporated into the standardized original sample sets to obtain an original data set D= {(x₁, y₁), (x₂, y₂), (x₃, y₃), ..., (x₈₂, y₈₂)}.

(4) The Pearson correlation coefficient between the feature values and the biodegradability of the organic molecules is calculated using the formula:

$$r = \frac{{\sum }_{i = 1}^{82} \left({x}_{i} - \overline{x}\right) \left({y}_{i} - \overline{y}\right)}{\sqrt{{\sum }_{i = 1}^{82} {\left({x}_{i} - \overline{x}\right)}^{2}} {\sum }_{i = 1}^{82} {\left({y}_{i} - \overline{y}\right)}^{2}}$$

where xᵢ is a feature value, yᵢ is a measured value of the biodegradability of the organic molecules in the sewage,

$$\overline{x} = \frac{1}{82} {\sum }_{i = 1}^{82} {x}_{i} , \overline{y} = \frac{1}{82} {\sum }_{i = 1}^{82} {y}_{i} .$$

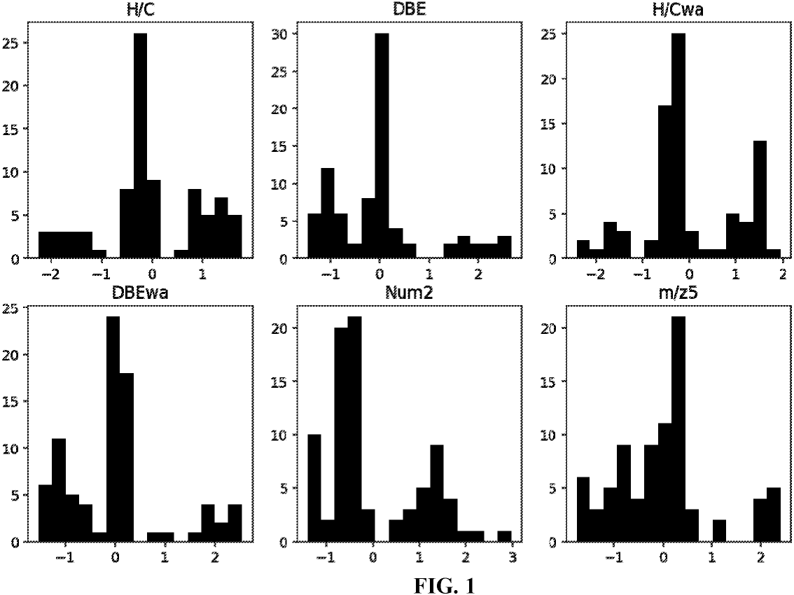

A correlation matrix of the feature values and the biodegradability of the organic molecules in the sewage is obtained using the formula. According to the absolute value of the correlation coefficient, the feature values (>0.7) highly correlated with the biodegradability of the organic molecules in the sewage are selected as input values of the neural network. The results show that there are six features that meet the selection criterion, including H/C, DBE, H/C_{wa}, DBE_{wa}, the number of protein/amino acid molecules Num2, and m/z5 for lignin molecules, as shown in FIG. 1.

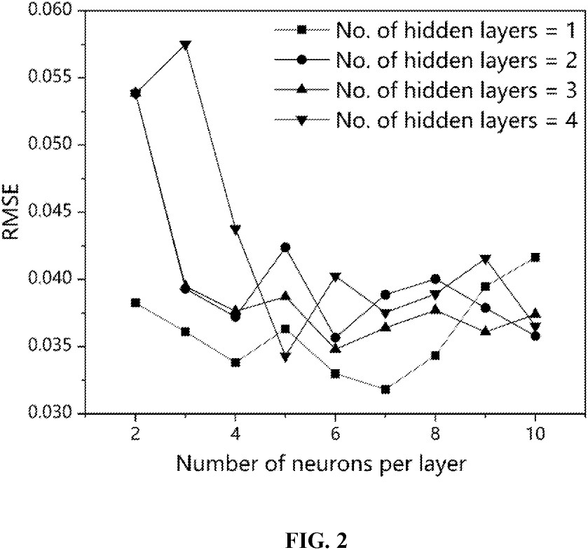

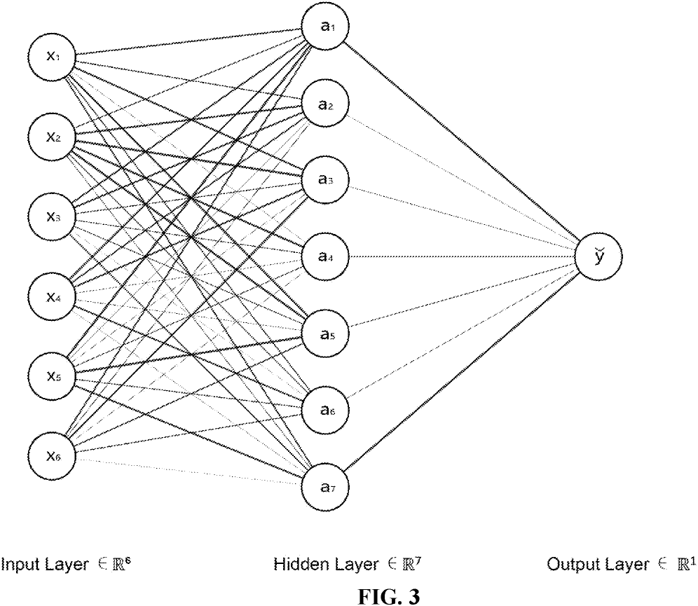

(5) The dataset is randomly split into a training set and a test set in a ratio of 7: 3. A model for biodegradability forecasting is established based on the multi-layer perceptron. The input value of the input layer is connected to the neurons in the hidden layer and the neurons in the hidden layer are connected to the neurons of the output layer. Each neuron in one layer is connected to all neurons in the next layer. The topology of the multi-layer perceptron is determined as follows: the numbers of the neurons in each hidden layer ranges from 2 to 10 according to the number of input variables; the number of the hidden layers ranges from 1 to 4 according to the characteristics of data structure; and the same number of neurons is used for all hidden layers. The RMSE of various topological structures set is shown in FIG. 2, and the model with lowest RMSE is selected, as shown in FIG. 3.

The topology of the multi-layer perceptron comprises six input neurons, seven hidden neurons, and one output neuron. The hyderparameters that need to be optimized comprises: an algorithm used to minimize the loss function, comprising stochastic gradient descent (SGD), adaptive moment estimation (Adam) and Limited-memory BFGS (L-BFGS); an activation function, comprising Sigmoid, tanh and ReLU; a parameter α∈(0.001,0.01) for L2 regularization term; and a maximum number iter ∈(50,200) of iterations. In this example, L-BFGS is selected to minimize the loss function due to its faster convergence speed; ReLU is selected as the activation function; the parameter α is set to 0.00223; and the maximum number iter is set to 65.

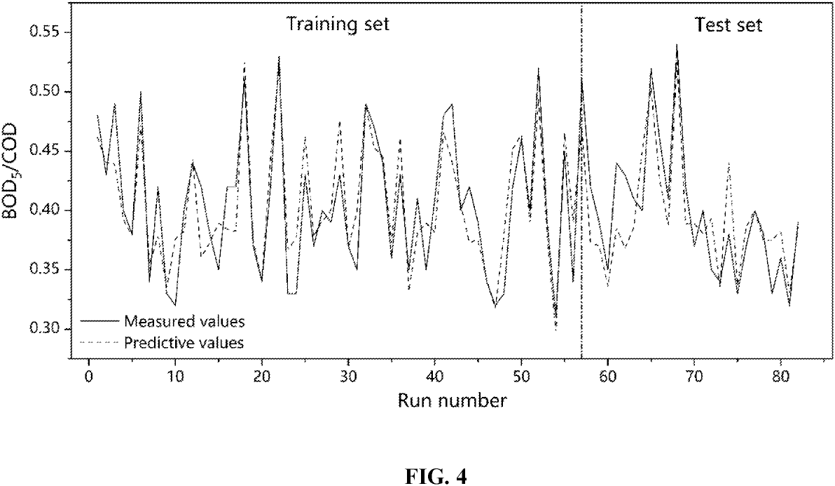

(6) The optimal hyperparameters are set before training the model. The performance of the trained model is tested on the test set, as shown in FIG. 4. The coefficient of determination (R²) and root mean squared error (RMSE) are used as indicators for evaluating the accuracy of the model.

(7) During training the model provides R² of 0.76 and RMSE of 2.85%; and during testing the model provides R² of 0.64 and RMSE 3.18%.

(8) The molecular composition information of organic molecules in the sewage sample is measured by a Fourier transform ion cyclotron resonance mass spectrometer.

(9) According to the requirements in (4), the desired feature values are extracted to obtain a feature vector X = (x₁; x₂; x₃; x₄; x₅; x₆); and, standardization is performed according to the mean values and variances of the respective feature values in the original data set to obtain a standardized feature vector X= (-0.332; 0.140; -0.354; 0.157; -0.564; 0.318)^{T}.

(10) The feature vector X is input into the trained prediction model, and the prediction model is run to obtain an output value of 0.37. The biodegradability of organic molecules in the sewage samples is 0.39. In accordance with the disclosure, there is no significant difference between the predicted value of the biodegradability of organic molecules in sewage, and the prediction accuracy is 94.9%.

### Example 2

Sewage samples from a sewage plant are selected to evaluate the biodegradability of organic molecules. The specific evaluation method was described below:

(1) The model establishment process is the same as that in Example 1.

(2) The molecular composition information of organic molecules in sewage measured by a Fourier transform ion cyclotron resonance mass spectrometer.

(3) The desired feature values are extracted to obtain a feature vector X = (x₁; x₂; x₃; x₄; x₅; x₆); and, standardization is performed according to the mean values and variances of the respective feature values in the original data set to obtain a standardized feature vector X = (-1.59; 1.81; -1.41; 1.80; -1.19; 2.00)^{T}.

The feature vector X is input into the trained prediction model, and the prediction model is run to obtain an output value of 0.34. The biodegradability of organic molecules in the sewage samples is 0.33. In accordance with the disclosure, there is no significant difference between the predicted value of the biodegradability of organic molecules in sewage, and the prediction accuracy is 97.0%.

It will be obvious to those skilled in the art that changes and modifications may be made, and therefore, the aim in the appended claims is to cover all such changes and modifications.

The invention claimed is:

1. A method, comprising:
(1) collecting molecular composition information and biodegradability data of organic molecules in a sewage sample;
(2) establishing a model for predicting biodegradability of organic molecules in sewage through machine learning;
(3) measuring the molecular composition information of organic molecules in sewage from a target sewage plant; and
(4) predicting, according to the model established in (2), the biodegradability of the organic molecules in the sewage from the target sewage plant.

2. The method of claim 1, wherein in (1), the molecular composition information of organic molecules in the sewage sample comes from data measured by a Fourier transform ion cyclotron resonance mass spectrometer, and the biodegradability of the organic molecules in the sewage is represented by BOD₅/COD.

3. The method of claim 1, wherein in (2), the model for predicting the biodegradability of organic molecules in the sewage is established by a multi-layer perceptron that is a neural network model used in machine learning, which comprises:
(a) calculating a molecular parameter of the organic molecules, and performing data standardization by using the molecular parameter as a feature value;
(b) calculating a Pearson correlation coefficient between the feature value and the biodegradability of the organic molecules; extracting, according to the absolute value of the Pearson correlation coefficient, desired feature values as input features in a neural network;
(c) splitting a dataset into a training set and a test set, determining topology of the neural network, which comprises a number of hidden layers and a number of neurons in each hidden layer; and
(d) optimizing hyperparameters of the model, training the neural network with the training set, and evaluating the performance of the neural network by using the test set.

4. The method of claim 3, wherein
in (a), the molecular parameter as the feature value comprises: molecular parameters of all organic molecules, and molecular parameters of seven classes of organic molecules;
the molecular parameters of all organic molecules comprise: a mass-to-charge ratio m/z, a number C of carbon atoms, a number H of hydrogen atoms, a number O of oxygen atoms, a number N of nitrogen atoms, a ratio O/C of the number of oxygen atoms to the number of carbon atoms, a ratio H/C of the number of hydrogen atoms to the number of carbon atoms, a number DBE of double bond equivalents, a ratio DBE/H of the number of double bond equivalents to the number of hydrogen atoms, a ratio DBE/O of the number of double bond equivalents to the number of oxygen atoms, a ratio (DBE-O)/C of a difference between the number of double bond equivalents and the number of oxygen atoms to the number of carbon atoms, an average value of a nominal oxidation state of carbon (NOSC) of all organic molecules, and strength weighted average values of molecular parameters, which are equal to a sum of products of respectively multiplying corresponding relative peak strength of molecules by m/z, C, H, O, N, O/C, H/C, DBE, DBE/H, DBE/O, (DBE-O)/C and NOSC;
the seven classes of organic molecules are: lipids, proteins/amino sugars, carbohydrates, unsaturated hydrocarbons, lignin, tannins and condensed aromatics; screening conditions for lipids are as follows: O/C < 0.2 and 1.7 < H/C < 2.2; screening conditions for proteins/amino sugars are as follows: 0.2 < O/C < 0.6, 1.5 < H/C < 2.2 and N/C ≥ 0.05; screening conditions for carbohydrates are as follows: 0.6 < O/C < 1.0 and 1.5 < H/C < 2.2; screening conditions for unsaturated hydrocarbons are as follows: O/C<0.1, 0.7<H/C<1.5; screening conditions for lignin are as follows: 0.1 < O/C < 0.6, 0.6 < H/C < 1.7, and a modified aromaticity index AImod < 0.67; screening conditions for tannins are as follows: 0.6 < O/C < 1.0, 0.5 < H/C < 1.5 and a modified aromaticity index AImod < 0.67; and, screening conditions for condensed aromatics are as follows: O/C < 1.0, 0.3 < H/C < 0.7 and the modified aromaticity index AImod ≥ 0.67; and
the molecular parameters of seven classes of organic molecules comprise: a mass-to-charge ratio m/zi, a number DBEi of double bond equivalents, and an average value of the nominal oxidation state of carbon NOSCi of seven classes of organic molecules, a proportion Numi of the number of molecules in each class, and strength weighted average values of molecular parameters, which are equal to a sum of products of respectively multiplying corresponding relative peak strength of molecules by m/zi, DBEi and NOSCi, which i represents the molecule classes.

5. The method of claim 3, wherein in (a), the data standardization is performed on the feature value using the formula:
$$z = \frac{\left(x - u\right)}{s}$$
where z is a standardized feature value, x is an original feature value, u is an average value of the features, and s is a standard deviation of the feature value.

6. The method of claim 3, wherein in (b), the Pearson correlation coefficient between the feature value and the biodegradability of the organic molecules is calculated using the formula:
$$r = \frac{{\sum }_{i = 1}^{n} \left({x}_{i} - \overline{x}\right) \left({y}_{i} - \overline{y}\right)}{\sqrt{{{\sum }_{i = 1}^{n} \left({x}_{i} - \overline{x}\right)}^{2}} {\sum }_{i = 1}^{n} {\left({y}_{i} - \overline{y}\right)}^{2}}$$
where xᵢ is a feature value, yᵢ is a measured value of the biodegradability of the organic molecules in the sewage,
$$\overline{x} = \frac{1}{n} {\sum }_{i = 1}^{n} {x}_{i} , \overline{y} =$$
$$\frac{1}{n} {\sum }_{i = 1}^{n} {y}_{i} ,$$
n is a total number of sewage samples; a correlation matrix of the feature value and the biodegradability of the organic molecules in the sewage is obtained using the formula; and according to the absolute value of the Pearson correlation coefficient, the feature value highly correlated with the biodegradability of the organic molecules in the sewage is selected as input values of the neural network.

7. The method of claim 3, wherein in (c), the dataset is randomly split into a training set and a test set in a ratio of 7: 3; a model for biodegradability forecasting is established based on the multi-layer perceptron; the input value of the input layer is connected to the neurons in the hidden layer and the neurons in the hidden layer are connected to the neurons of an output layer; each neuron in one layer is connected to all neurons in a next layer; the topology of the multi-layer perceptron is determined as follows: a range for the numbers of the neurons in each hidden layer is determined according to the number of input variables; a range for the number of the hidden layers is determined according to the characteristics of data structure, and the same number of neurons is used for all hidden layers.

8. The method of claim 3, wherein in (d), the topology of the multi-layer perceptron comprises m input neurons, n hidden neurons, and one output neuron; an output of the neural network, a predicted value for biodegradability of sewage, is expressed within the following equation:
$${y}^{⌣} = W θ \left({\sum }_{i = 1}^{m} {w}_{i} {x}_{i} + {b}_{1}\right) + {b}_{2}$$
where, y̌ is a predicted value, W and wᵢ are weights of a hidden layer and an input layer, respectively; b₁ and b are bias added for the hidden layer and the output layer, respectively; and θ is a an activation function;
training the neural network is to minimize a loss function; the loss function is expressed as below:
$$L o s s = \frac{1}{2} {\left‖{y}^{⌣} - y\right‖}_{2}^{2} + \frac{α}{2} {\left‖W\right‖}_{2}^{2}$$
where,
$$α / 2 {\left‖W\right‖}_{2}^{2}$$
is a L2 regularization term for penalizing a complex model;
parameters in an opposite direction of a gradient of an objective function are updated at each iteration through gradient descent; an example formula of gradient descent is as follows where the weights are updated:
$${W}^{i + 1} ← {W}^{i} + Δ {W}^{i}$$
$$Δ {W}^{i} = - η ∇ L o s {s}_{W}^{i}$$
where, i is a number of iterations, η∈(0,1) is a learning rate, and
$$∇ L o s {s}_{W}^{i}$$
is a gradient of the loss function with respect to the weights.

9. The method of claim 8, wherein the hyderparameters that need to be optimized comprises: an algorithm used to minimize the loss function, comprising stochastic gradient descent (SGD), adaptive moment estimation (Adam) and Limited-memory BFGS (L-BFGS); an activation function, comprising Sigmoid, tanh and ReLU; a parameter a for L2 regularization term; and a maximum number iter of iterations; the training set is used to fit the neural network, and the test set is used to measure the performance of the neural work; a coefficient of determination (R²) and root mean squared error (RMSE) are used as indicators for evaluating the accuracy of the model; and R² is calculated using the formula:
$${R}^{2} \left(y , {y}^{⌣}\right) = 1 - \frac{{\sum }_{i = 1}^{n} {\left({y}_{i} - {y}^{⌣}\right)}^{2}}{{\sum }_{i = 1}^{n} {\left({y}_{i} - \overline{y}\right)}^{2}}$$
RMSE is calculated using the formula:
$$R M S E = \sqrt{\frac{1}{n} {\sum }_{i = 1}^{n} {\left({{y}^{⌣}}_{i} - {y}_{i}\right)}^{2}}$$
where, yᵢ is a measured value, y̌ᵢ is a predicted value,
$$\overline{y} = \frac{1}{n} {\sum }_{i = 1}^{n} {y}_{i} ,$$
n is a total number of the sewage sample.

10. The method of claim 1, wherein in (3) and (4), using the model to predict the biodegradability of the sewage comprises:
(a) measuring the molecular composition information of organic molecules in the sewage sample by a Fourier transform ion cyclotron resonance mass spectrometer;
(b) extracting a desired feature value; and performing data normalization on the feature value; and
(c) feeding the feature value in (b) into the model, running the model to obtain an output value for the biodegradability of organic molecules in sewage.
